(19) United States
(12) Patent Application Publication
(10) Pub. No.: US 2004/0067502 A1
(43) Pub. Date: Apr. 8, 2004

(54) Multiplex assays using nanoparticles

(76) Inventors: Rolf Guenther, Hamburg (DE); Guenter Bauer, Schmalfeld (DE); Franz-Josef Meyes-Almes, Iserlohn (DE); Guenther Fuhr, Berlin (DE)

Correspondence Address: JACOBSON HOLMAN PLLC, 400 SEVENTH STREET N.W., SUITE 600, WASHINGTON, DC, 20004, US

(21) Appl. No.: 10/398,512

(22) PCT Filed: Oct. 11, 2001
(86) PCT No.: PCT/EP01/11765

(30) Foreign Application Priority Data
Oct. 11, 2000 (EP) 00122070.6
Oct. 11, 2000 (EP) 00122071.4

(51) Int. Cl. C12Q 1/68; G01N 33/53

(52) U.S. Cl. 435/6; 435/7.1

(57) ABSTRACT

A method for detecting the binding between binding sites, comprising the following steps: providing a population of nanoparticles N₁, . . . Nₓ, each nanoparticle having at least one characteristic electrical field property and at least one first binding site, wherein the characteristic electrical field property codes for the corresponding first binding site of each nanoparticle, adding a sample to be analyzed which possibly comprises at least oen analyte with at least one second binding site capable to bind to said first binding site, wherein said analyte and/or said nanoparticles N₁, . . . or Nₓ have at least one detectable property which changes upon binding of said first binding site to said second binding site, the detectable property being different from the electrical field property, applying an electrical field, separating said population of nanoparticles with regard to said electrical field property, measuring the presence or absence of a change of the at least one detectable property.

The present invention relates to a method for detecting the binding between binding sites of molecules and nanoparticles. Especially, it relates to multiplex assays using nanoparticles with selected electric and/or dielectric properties and to nanoparticles with selected electric and/or dielectric properties.

Especially in diagnosis, drug discovery or therapy it is important to detect and characterise quantitatively or qualitatively a wide variety of physiologically active compounds, both naturally occurring or synthetic, in a short time.

Therefore, it is advantageous to have methods, which are suitable for th measurement of several parameters of a sample at the same time, such as e.g, the simultaneous determination and characterization of several molecules, e.g. analytes, possibly contained in a sample solution, e.g. a biological sample. In general methods for the determination and/or characterization of analytes are called assays.

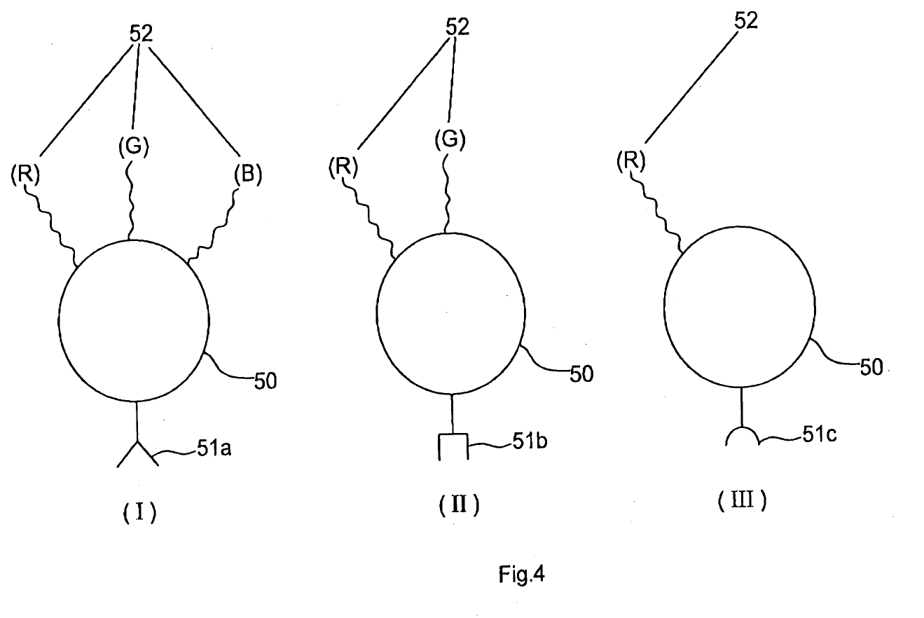

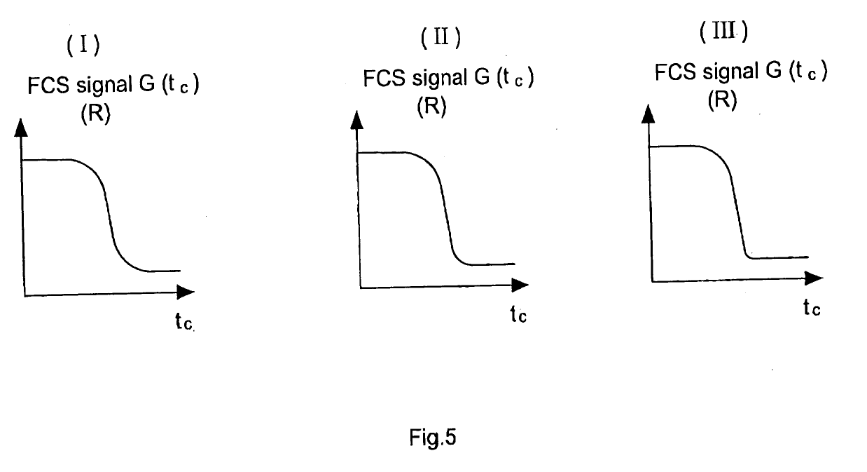

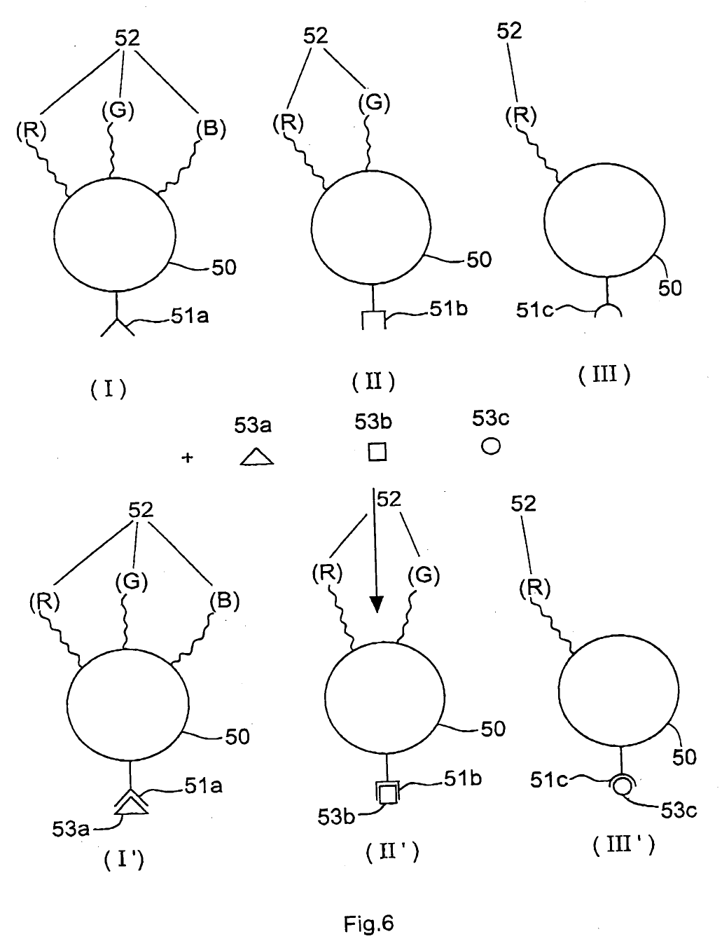

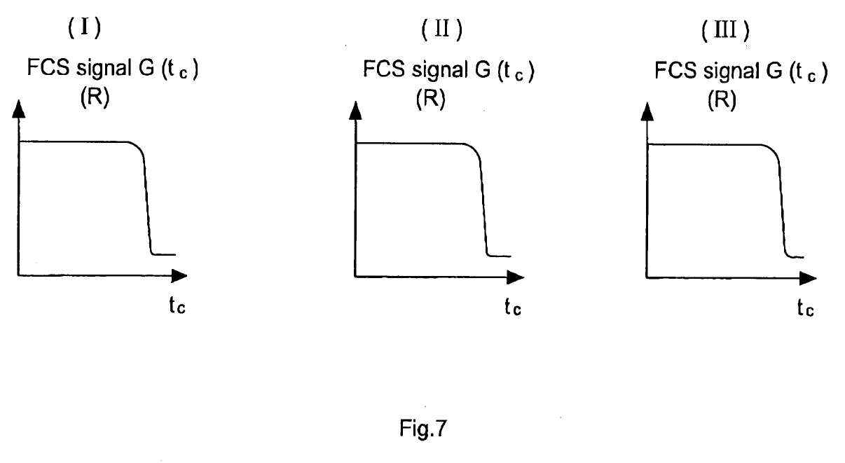

However, the assays for the simultaneous measurement of several parameters also called multiplex assays, are difficult to perform. Assays are generally based on the specific binding of at least two binding partners, whereas one partner often is labeled. The presence of an analyte in the sample to be characterized therefore leads to th forming of a complex between the analyte and a labeled binding partner. The simultaneous determination of different analytes in one solution requires the sinultaneous detection of different complexes in the same solution. This is only possible if for each kind of these complexes a specific label is present. For a multiplex fluorescence assay this means that for each fluorescence label a separate laser and a separate detector are necessary to distinguish the different complexes (binding pairs). An example of such assays is illustrated in FIGS. 4 to 7. FIG. 4 shows an FCS assay of three differently labeled particles, e.g. synthetic beads. The first bead is labeled with a red (R), green (G) and blue (B) marker, the second bead with a red (R) and a green (G) label and the third bead with a red (R) marker, whereas each specific labelling is called a coding scheme. Each type of bead additionally comprises a characteristic binding site able to bind selectively to the binding site of sample components, e.g. analytes such as antibodies, or antigens. It is not possible to distinguish between the different types of beads by detecting only the fluorescence of the red label, as shown in FIG. 5. However, it is necessary to use three different light sources and detectors each for each kind of label. The same problem arises when the different sample components have bound to the particles, and complexes are formed as shown in FIG. 6. In order to be able to detect the binding between each type of bead an the characteristic sample component it is not sufficient to detect the fluorescence of the red label (FIG. 7).

Another problem with regard to the characterization of a sample, especially of a biological sample, is the restricted availability of biological samples, their often very small amounts accessible and the low concentrations of analytes in such samples.

Therefore, to reduce the amount of sample needed especially in biological assays, assays are often performed in microfluidic and/or microstructured devices. But the known methods, show significant disadvantages which limit the wide applicability of the assays in daily use. For example, the diffusion of the sample in the microstructured cavities and channels of the microstructures sets an upper limit for incubation times possible in the assays. This means, that e.g. the period of time an enzyme used in a reaction has to turn its substrate to a substantial amount of product is limited and the product is to be measured in presence of large amounts of substrate.

As mentioned above the concentrations of analytes in the biological samples are often very low and the quantity of such samples being available is small. Therefore, despite of the miniaturisation, a better sensitivity of the methods is important. To enhance sensitivity, electrophoretic separation of substrate and product (e.g. complex) prior to detection of the product is performed, to measure the product absolutely separately and not in the presence of the other assay components such as substrate. However, this method is only suited for a small number of assays, because the separation requires a difference in electronic properties between th assay components, e.g. the substrate and the product.

To enhance the separation of the assay components beads on which some of the assay components are immobilized are used. However, beads are usually difficult to handle since they tend to sediment and therefore require constant agitation. This is especially a problem in microfluidic and/or microstructured devices, where external access for agitation devices is difficult to provide. Furthermore, beads may clog capillaries in said devices and therefore cause severe risk of dysfunction.

The use of magnetic beads, which are also used in macroscopic systems to separate assay components by virtue of magnetic forces before detection are, however, also difficult to apply in microfluidic and/or microstructured devices as the generation of strong magnetic fields requires bulky sources, such as ferromagnetic tools (which cannot be switched) or electromagnetic coils, which are very difficult to design in a microfluidic and/or microstructured device.

Therefore, it is the object of the present invention to provide a method for the simultaneous detection of the binding between different binding sites, which does not show the above mentioned disadvantages. A method which is highly sensitive, which does not show sedimentation of the particles, which allows the examination of a population of different particles and allows the use of a simple experimental set-up, and which provides reliable data on the binding event is necessary. It is also an aspect of the present invention to provide particles to be used in such method.

The object is achieved by the method described in claim 1 and the nanoparticle described in claim 25. Preferred embodiments are indicated in the dependent claims.

The present invention provides a method for detecting the binding between binding sit s, comprising the following steps.

providing a population of nanoparticles N₁, . . . Nₓ, each nanoparticle having at least one characteristic electrical field property and at least one first binding site, wherein the characteristic electrical field property codes for the corresponding first binding site of each nanoparticle,

adding a sample to be analyzed which possibly comprises at least one analyte with at least one second binding site capable to bind, preferably selectively bind, to said first binding site, wherein said analyte and/or said nanoparticles N₁, . . . or Nₓ have at least one detectable property which changes upon binding of said first binding site to said second binding site, the detectable property being different from the electrical field property,

applying an electrical field,

separating said population of nanoparticles with regard to said electrical field property,

measuring the presence or absence of a change of the at least one detectable property.

It is possible according to the present invention that the electrical field is applied in the form of a field gradient.

According to the inventive method it is preferred that e.g. all the nanoparticles of type N₁ show the electric property E₁, all the nanoparticles of type N₂ show the electric property E₂, etc. The characteristic electrical field property codes for the corresponding first binding site. In the aforementioned example this means that all nanoparticles of type N₁ have a first binding site B₁, all the nanoparticles of type N₂ have a first binding site B₂, etc.

The measurement of the presence or absence of the change of said detectable property is preferably performed simultaneously with or after applying the electrical field.

Within the present invention, the nanoparticles comprising said first binding site and said analytes comprising said second binding site selectively bind to each other to form a complex, wherein after the complex formation the presence of change of said detectable property can be detected. This means, that preferably the detectable property changes due to the binding event and this change is detected.

Preferably, said first binding site comprises an assay component, especially an antigen, an antibody, an enzyme, a receptor, a ligand, DNA, RNA, a receptor, a dendrimer and/or a small peptide.

In other preferred embodiments, each of said nanoparticles N₁ . . . or Nₓ comprises at least one detectable label having said detectable property. More preferably, one single detectable label is used which is identical for all nanoparticles N₁ . . . or Nₓ. Thus, it is possible e.g. to use one single laser for excitation of fluorescence of this label and one single detector to measure the fluorescence signal.

It might also be preferred that each of said analytes comprises at least one detectable label having said detectable property. More preferably, each analyte comprises one single detectable label is used which is identical for all analytes. Thus, it is possible e.g. to use one single laser for excitation of fluorescence of this label and one single detector to measure the fluorescence signal.

Due to the fact that it is possible to use e.g. one single excitation light source, the setups to be used are relatively simple and do not require the use of several expensive light sources and detectors. This is also true for other detection methods.

In preferred embodiments of the present invention the label is a magnetic label and/or a luminescence label, especially a fluorescence label, and/or a radioactive label.

Additionally, it might be preferred that the presence or absence of change of said detectable property is measured by detecting radioactivity and/or magnetism and/or absorption and/or colorimetry and/or luminescence, especially fluorescence spectroscopy, in particular by measuring fluorescence intensity (Fl) and/or fluorescence polarisation (FP) and/or fluorescence lifetime (FLT), and/or (cc)fluorescence correlation spectroscopy and/or fluorescence intensity distribution analysis (FIDA), 2D-FIDA, and/or cFLA and/or FILDA and/or GMR (Giant Magneto Resistance) and/or GMI (Giant Magneto-Impedance).

These methods allow the sensitive and reliable determination of the binding of the different binding sites.

The nanoparticles N₁, . . . or Nₓ used in the inventive method preferably comprise at least one charged molecule which determines their electrical field property and which is bound to the surface of said nanoparticles N₁, . . . or Nₓ.

Whereas charged molecule means molecule comprising a charge and/or molecule on which a charge is induced by the electric field provided.

For example all particles of type N₁ show charged molecules of type A and all particles of type N₂ show charged molecules of type B₂, etc.

It is possible that cavities can be found in the nanoparticles. The charged molecules could also be found in cavities.

Preferably, said charged molecule is different from said first binding site and/or said second binding site.

The charged molecule is for xample selected from the following group of molecules PO₄³⁻, SO₄²⁻, NH₃⁺, NH₂, DNA or oligonucleotides (perrnanent and induced dipole moment), highly charged proteins, sugars, starch or organic polymers especially polyamines, polycarboxylates, especially, “Babyabsorber”, polyacrylate, PEG backbones, polyvinylalcohols, branched polymers, glass and/or polystyrene and/or metaloxide and/or organic polymer and/or supramolecular units, such as dendrimers or fullerenes and/or DNA and/or RNA and/or small peptides.

It is further preferred, that a plurality of charged molecules form a shell-like film on the surface of said nanoparticles N₁, . . . or Nₓ. This shell-like film could be applied to existing nanoparticles not having the desirous characteristic electric properties to be used in the method of the present invention.

For example all particles of type N₁ show a shell-like film of type A and all particles of type N₂ show a shell-like film of type B, etc.

It is also preferred that a plurality of charged molecules form at least two shell-like films on the surface of said nanoparticles N₁, . . . , or Nₓ which might be the same or different.

In the inventive method it is preferred that the electric field property is the susceptibility, and the susceptibility of said nanoparticles N₁, . . . or Nₓ is determined by the number and/or kind of charged molecules.

It is further preferred that the susceptibility is the AC susceptibility and/or DC susceptibility, and/or AC dielectric susceptibility and/or DC dielectric susceptibility.

For the measurements it may be further preferred that said electrical field properties of said nanoparticles N₁, . . . or Nₓ change upon binding of the first binding site to the second binding site. This change may have a positive effect on the separation step, because it may increase the differences of the field properties between at least two different nanoparticles.

But R may also be preferred that said electrical field properties of said nanoparticles N₁, . . . or Nₓ are not, or are only slightly changed upon binding of the first binding site to the second binding site. Therefore, e.g. the susceptibilities of such nanoparticles do not change upon binding of the analyte.

According to the invention it may also be preferred that the electrical field exercises forces on said nanoparticles N₁, . . . or Nₓ which result in a net motion of the nanoparticles N₁, . . . or Nₓ relatively to each other.

In one embodiment according to the inventive method at least two nanoparticles N₁ and N₂ with different characteristic electrical field properties are used.

It may be also preferred that the nanoparticles N₁, . . . or Nₓ are moved by the electrical field to an area where in addition to the electrical field an additional field is applied, preferably a field based on hydrostatic pressure and/or a second electrical field.

It is possible, to apply hydrostatic pressure to the device especially microstructured and/or microfluidic devices such as microchannels, wherein the method is performed, to support the separation of the nanoparticles due to the electrical field and therefore to support the selectivity of the method.

The hydrostatic pressure preferably exerts a force on said nanoparticles N₁, . . . or Nₓ in the field area, wherein the electrical field is superimposed on said hydrostatic pressure, so that a net force results which is different for the nanoparticles N₁, . . . . or Nₓ relatively to each other.

According to the present invention said hydrostatic pressure may intentionally switched on or off, especially by internal and/or external valves and/or pistons.

It may be also preferred that said electrical field is also intentionally switched on or off, especially correlated proportionally and/or otherwise correlated to said switch of said hydrostatic pressure.

At may also be preferred that the frequency of said electrical field, especially AC field, is varied, especially swept.

The switches may also be set related to a sensor signal indicating the presence or arrival of a sample to be characterised in said field area.

In a preferred embodiment the field area is a capillary with a diameter of <500 microns, especially of <100 microns and/or the field area may be an open section in a channel on a chip.

In this assay the said electrical field exercises force may define a confinement of the field area, especially a field area shaped as a capillary, wherein said capillary may contain sideward connections to and/or intersections with fluid reservoirs, especially further capillaries.

According to the present invention said detectable properties of said nanoparticles N₁ . . . or Nₓ are measured while passing of said nanoparticles N₁, . . . or Nₓ along a stationary detector and/or by scanning the individual positions of said nanoparticles N₁, . . . or Nₓ sequentially with a detector and/or simultaneously by using a detector array.

It is further preferred that according to the inventive method said nanoparticles N₁, or Nₓ are nanoparticles with a diameter of less than 10 microns, preferably less than 1 microns, more preferably less than 200 nanometers, most preferably less than 100 nanometers.

In generall the method of the present invention can e.g. be used to qualitatively and/or quantitatively determine different analytes in a sample.

It can especially be used to perform miniaturized assays, especially in microfluidic and/or microstructured devices wherein nanoparticles are used which e.g. have defined charges and/or DC (electric) and/or AC (dielectric) susceptibilities, and which are referred in the following in general to as characteristic electrical field properties.

Preferably, as mentioned above the method of the present invention may be performed using microfluidic and/or microstuctured devices, more preferably chip-systems. The devices can comprise one or more entrance microchannels, a system of microchannels e.g. capillaries, for storing, processing, or transporting fluids, suspensions or solutions, one or more exit microchannels and optionally cavities, reservoirs, e.g. for the storage of solutions for washing, cultivating, conservation, cryo-conservation or for the storage of the separated particles, pumps, e.g. micropumps, peristaltic pumps syringe pumps, electroosmotic fluid and particle transporting devices, and sensors for the determination of sample properties, e.g. temperature, pH, or sensors to determine the arrival of the individual particles, in order to turn on the electrical field and/or the hydrostatic pressure and/or the detector. Additionally, it can comprise at least one detector, preferably one single detector, to measure the detectable properties, means to provide the electrical field for separation, and means to provide hydrostatic pressure. Preferably, the microchannels have diameters of less than 500 microns, mor preferably less than 100 microns. In one preferred embodiment, the area where the electrical field is applied, meaning the chip-system is an open section and the dielectric field forces define the different microchannels. Using a chip-system with a system of different microchannels it is possible, that during the performance of an assay, the different assay components can be introduced at different points of the chip-systems. This is especially important, 1f at a given stage of the assay some components should not be present because they would interfere. The chip-system can further comprise valves, membranes, or ion barriers.

In other preferred embodiments, the chip-system can comprise further means for manipulation and separation of the particles, fluids, suspensions and/or solutions. These means can include additional microelectrodes (i) to produce electrical fields, especially dielectrical field cages, to hold individual particles or (ii) to produce field barriers to direct the particles into different microchannels. It can further comprise optical tweezers, e.g. laser tweezers.

Preferably, such chip-systems are made of silicone or other polymeric material.

An example for an assay performed in a microsystem, e.g. chip-system is given in the following:

According to the present invention it may be preferred that said nanoparticles N₁, . . . or Nₓ comprise an enzyme and a possible assay in a microstructured device may be performed comprising the following steps:

injection of assay components into said device, especially of substrate for said enzyme, into the field area of the microsystem,

addition of said nanoparticles comprising said enzyme by one of the fluidic connections,

transport of said nanoparticles along said capillary to a second of said fluidic connections by applying an electrical and/or hydrostatic force, especially by hydrostatic pressure,

removal of said nanopartides N₁, . . . or Nₓ comprising the enzyme at said second connection by applying an electrical and/or hydrostatic force, especially by electric, preferred dielectric forces.

In other preferred embodiments an incubation of the assay components and the nanoparticles is performed.

In such microsystems it is possible to bring e.g assay components which are not desired to be present during the complete assay to the field area to the assay at said cross sections.

According to the invention electrical and/or hydrostatic forces are used to separate the assay components e.g. nanoparticles N₁, . . . or Nₓ and/or analytes specficly.

It may be preferred that the frequency of said electrical field, especially AC field, is varied, especially swept. The force executed may be varied intentionally over time, so that the flow velocity between the interconnections of the microsystem may be varied accordingly, and that said additional forces at the connections may be adjusted accordingly.

It may also be preferred that the concentration of at least one of said nanoparticles N₁, . . . or Nₓ and/or analytes is varied intentionally over time.

It is preferred that the time evolution of said concentrations is measured to determine kinetics of said enzymatic reaction and/or titration of said assay components, e.g. analytes.

According to the inventive method at least one detection position is present in said capillary in the microsystem, especially behind one of the connections, and wherein serial measurements are executed to determine parameters of said assay, especially signals relating to the concentration of the analytes.

One advantage of the inventive method is, that the enzyme bound to a nanoparticle may be directly transferred to the substrate e.g. present in a capillary and/or reservoir of the microstructured device by the applying an electrical field.

Nanoparticles N₁, . . . or Nₓ according to the present invention preferably have a diameter of less than 10 microns, preferably less than 1 microns, more preferably less than 200 nanometers most preferably less than 100 nanometers so that they do not sediment in solution. They preferably comprise glass and/or polystyrene and/or polypropylene and/or metaloxide and/or organic polymer and/or supramolecular units, such as dendrimers or fullerenes and/or DNA and/or RNA and/or small peptides.

In a further aspect of the present invention nanoparticles may be coated with molecules such as PO₄³⁻, SO₄²⁻, NH₃⁺, NH₂, DNA and/or oligonucleotides (permanent and induced dipole moment), highly charged proteins, sugars, starch, organic polymers especially polyamines, polycarboxylates (“Babyabsorber” Polyacrylat, PEG backbones, polyvinylalcohols, branched polymers, dendrimers) glass and/or polystyrene and/or polypropylene and/or metaloxide and/or organic polymer and/or supramolecular units, such as dendrimers or fullerenes and/or DNA and/or RNA and/or small peptides.

According to the present invention said first binding site of the nanoparticles N₁, . . . or Nₓ comprise an assay component, especially an antigen, an antibody, an enzyme, a receptor, a ligand, DNA, RNA, a ligand, a receptor, a dendrimer and/or a small peptide.

The nanoparticles N₁, . . . or Nₓ according to the present invention comprise at least one charged molecule which determines ist electrical field property, wherein said charged molecule is different from said first binding site.

According to the present invention the nanoparticles N₁, . . . or Nₓ may comprise a plurality of charged molecules, which form a shell like film on the surface of said nanoparticles N₁, . . . or Nₓ.

The nanoparticles N₁, . . . or Nₓ are preferably coated by at least two shell-like films which might be the same or different. More preferably, the films are different.

It might be preferred, that the electric field property is the susceptibility, wherein the susceptibility of said nanoparticles N₁, . . . or Nₓ is determined by the number and/or kind of charged molecules.

It might be further preferred that the susceptibility is the AC susceptibility and/or DC susceptibility and/or AC dielectric susceptibility and/or DC dielectric susceptibility. Preferably, the nanoparticles and the shell-like films show large differences in their dielectric constants and/or conductivity. Preferably, there are also large differences in these parameters e.g. between the shell-like film and the assay solution. E.g. the dielectric constant of the bead may be 20, and of the shell-like film 80. The shell-like films may for example preferably comprise DNA. In one preferred embodiment, the dielectric constant and conductivity are related to DC excitation, especially in the MHz and KHz region.

The electrical field properties of said nanoparticl s N₁, . . . or Nₓ might change upon binding of the first binding site to the second binding site. It might be further preferred that the electrical field prop rties of said nanoparticles N₁, . . . or Nₓ are preferably not, or only slightly changed upon binding of the first binding site to the second binding site.

The electrical field applied in the method of the present invention exercises forces on said nanoparticles N₁, . . . or Nₓ which result in a net motion of the nanoparticles N₁, . . . or Nₓ relatively to each other.

This will be further explained using the following example. A sample is provided, containing 3 different analytes A₁, A₂ and A₃. e.g. 3 different antigens, with second binding sites B₁, B₂ and B₃. Additionally, a population of nanoparticles of N₁, N₂, N₃ is provided. N₁, N₂ and N₃ have characteristic electrical field properties E₁, E₂ and E₃ and carry second first binding sites SB₁, SB₂ and SB₃, preferably different antibodies. The first binding sites bind selectively to the corresponding second binding sites e.g. by the formation of complexes. When applying the electrical field, the different complexes move differently due to their characteristic electrical properties. This is regarded as a relative motion of the nanoparticles with respect to each other.

It might be preferred that at least two nanoparticles with different electrical field properties are used in the method of the present invention.

Additionally, it might be preferred that nanoparticles N₁, . . . or Nₓ are moved by the electrical field to an area where in addition to the electrical field, an additional field is applied, preferably a field based on hydrostatic pressure and/or a second electrical field.

The detectable properties of said nanoparticles N₁, . . . or Nₓ are preferably measured while passing along a stationary detector and/or by scanning the individual positions of said nanoparticles N₁, . . . or Nₓ sequentially with a detector and/or simultaneously by using a detector array.

Preferably, one nanoparticle N₁, . . . or Nₓ comprises one single detectable property which is identical for all nanoparticle N₁, . . . or Nₓ. Therefore, it is possible to detect all types of analytes e.g. using one excitation laser and one detector.

Nanoparticles according to the present invention may also only consist of DNA and/or RNA molecules and/or small peptides which are labeled preferably by magnetic labels and/or fluorescence labels and/or radioactive labels.

The nanoparticies according to the present invention are very useful especially in assays in microstructured and/or microfluidic devices, preferably in multiplex assays. Whereas multiplex assays shall mean a variety of different assays in parallel in one solution or the determination of different analytes/substances in one sample in parallel. It is for example possible to create nanoparticles, which are each labeled with the same label, e.g. dye, especially a fluorescence dye, but which each have different dielectric properties and/or electric susceptibilities and/or charges. Each kind of these nanoparticles may have bound a different assay component, e.g. a binding ligand and/or receptor on its surface or be itself an assay component such as nanoparticles consisting of DNA and/or RNA and/or peptides and/or dendrimers, After adding a sample to be tested to a mixture of said nanoparticles and after incubation and preferably binding of sample components, e.g. analytes to the nanoparticles, the nanoparticles are preferably separated by their different dielectric properties and/or electric susceptibilities and/or charges and then or in parallel the detectable property of the detectable label, e.g. luminescence, e.g. fluorescence and/or and/or magnetic properties and/or radioactivity is detected separately for any type of nanoparticles. Because of the separation of the different nanoparticles by their behaviour in the electrical field only at least one type of label, e.g. dye, is sufficient to detect a possible molecule, e.g. analyte, binding to any of the nanoparticles. Therefore, the detection unit may be very simple as well.

The present invention will become better understood and other aspects, advantages and objectives of the present invention will become apparent from th description taken in close conjunction with the accompanying Figures. These are for illustration only, and thus are not to be considered as limiting the present invention. Changes and modifications within the spirit and scope of the invention will become apparent to those skilled in the art from the description and the Figures.

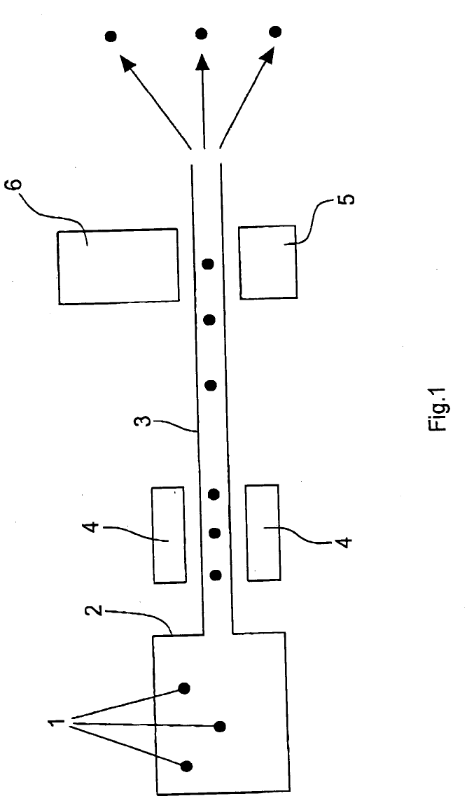

FIG. 1 shows a chip-system for detecting nanoparticles in a microchannel,

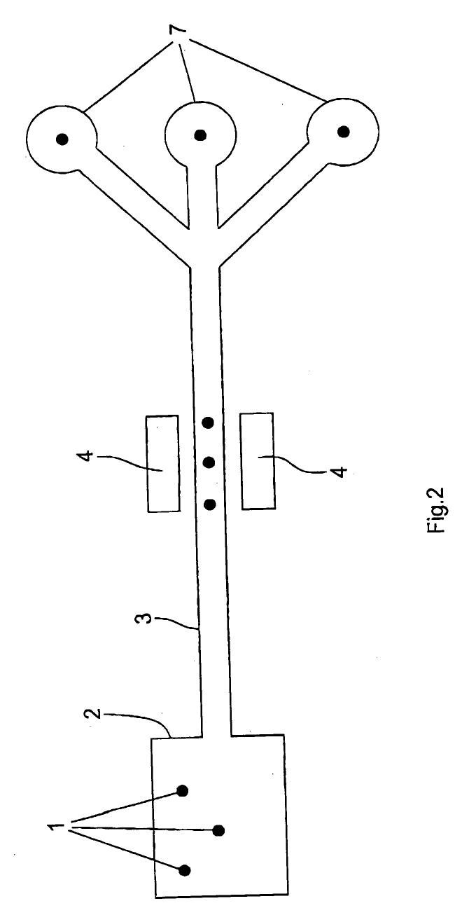

FIG. 2 shows a chip-system for detecting nanoparticles in separate reservoirs,

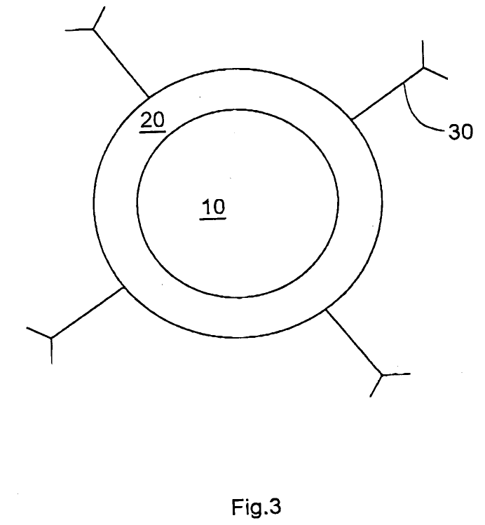

FIG. 3 shows a schematic drawing of one type of nanoparticle according to the invention,

FIG. 4 shows a prior art example using three differently labeled particles,

FIG. 5 shows that the impossibleness to distinguish the three labeled particles by detecting the red fluorescence only.

FIG. 6 shows the prior art example of FIG. 4. after binding of the characteristic binding partners, and

FIG. 7 shows that the impossibleness to distinguish three differently labeled complexes by detecting the red fluorescence only.

FIG. 1 can be described as follows. Complexes (1) to be detected might for example be formed in a reservoir (2) by the selective binding of the first binding site on the nanoparticles N₁, . . . or Nₓ (e.g. several different antibodies) to the second binding site on the analytes (e.g. several different antigens). The complexes formed which are comprising the nanoparticles are introduced into the microchannel (3) from the reservoir (2) e.g. by using micropumps (not shown) In the microchannel (3) an electrical field is applied using electrical field generators (4), separating the complexes du the different characteristic electrical properties of the nanoparticles comprised in the complexes. After the separation of the complexes the change of the detectable property due to the binding event, e.g. the change in fluorescence, is measured by a single detector (5) after applying e.g. a single laser (6) for excitation. After the measurement of the detectable property the individual types of complexes, meaning the different types of nanoparticles, can be distributed to different reservoirs e.g. by using electrical barriers (not shown) as indicated in FIG. 1. In other embodiments, the nanoparticles are scanned by a moving detector at their individual positions and/or the individual positions, where said nanoparticles are present, are studied simultaneously, especially by using an array detector.

Another possibility to be used for the method of the present invention is the chip-system shown in FIG. 2. As described above, complexes (1) to be detected might for example be formed in a reservoir (2) by the selective binding of the first binding site on the nanoparticles (e.g. several different antibodies) to the second binding site on the analytes (e.g. several different antigens). The complexes formed which are comprising the nanoparticles are introduced into the microchannel (3) from the reservoir (2) e.g. by using micropumps (not shown). In the microchannel (3) an electrical field is applied using electrical field generators (4), separating the complexes due the characteristic electrical properties of the nanoparticles comprised in the complexes. After the separation, the different fractions of complexes are collected in separate storage reservoirs (7). The distribution of the complexes to the different reservoirs (7) can e.g. be achieved using electical barriers at the cross-section (not shown). The measurement of the change of the detectable property is then performed in the different reservoirs (7) using a moving detector at the individual reservoir positions and/or the individual reservoirs, where said nanoparticles are present, are studied simultaneously, especially by using an array detector. The excitation light source and the detector is not shown in this Figure.

It is possible, to apply also hydrostatic pressure to the microchannels to support the separation of the nanoparticles due to the electrical field.

FIG. 3 shows a schematic drawing of one type of nanoparticle according to the invention.

Nanoparticles according to the present invention (10) preferably have a diameter of less than 10 microns, preferably less than 1 microns, more preferably less than 200 nanometers most preferably less than 100 nanometers so that they do not sediment in the microfluidic and/or microstructured devices. They preferably comprise glass and/or polystyrene and/or polypropylene and/or metaloxide and/or organic polymer and/or supramolecular units, such as dendrimers or fullerenes and/or DNA and/or RNA and/or small peptides and/or labels preferably magnetic labels and/or fluorescence labels and/or radioactive labels.

In a further aspect of the present invention nanoparticles may be coated with charged molecules (20) such as PO₄³⁻, SO₄²⁻, NH₃₊, NH₂, DNA and/or oligonucleotides (permanent and induced dipole moment), highly charged proteins, sugars, starch, organic polymers especially polyamines, polycarboxylates (“Babyabsorber” polyacrylate, PEG backbones, polyvinylalcohols, branched polymers, dendrimers) and/or shell-like films (20) to define selected electrical field properties. Preferably, the nanoparticles and the shell-like films show large differences in their dielectric constants and/or conductivity. Preferably, there are also large differences in these parameters between the shell-like film and the assay solution. E.g. the dielectric constant of the bead may be 20, and of the shell-like film 80. The shell-like films may for example preferably comprise DNA. In one preferred embodiment, the dielectric constant and conductivity are related to DC excitation, especially in the MHz and KHz region.

Furthermore on the outer surface of these nanoparticles preferably binding sites (30), such as molecules (30) and/or ligands (30) and/or receptors (30) are bound which may be labeled preferably by a fluorescence label and/or radioactive label and/or polymer and/or peptide.

FIGS. 4 to 7 show an example from the prior art. Three different beads (50) (I, 11, 111) with three different binding sites on the surface (51a, 51b, 51c) and red (R), green (G) and blue (B) fluorescent markers (labels) (52) (FIG. 4) are used. FIG. 5 shows the FCS signal for the red marker (R) for all three beads (I, II, III). The signals measured are identical for all particles (I, II, III), indicating, that it is not possible to distinguish between the different beads by measuring the red fluorescence only. The binding sites (51a, 51b, 51c) bind selectively to the corresponding bindings sites (53a, 53b, 53c) of the analyte to form complexes (I′, II′, III′) (FIG. 6). FIG. 7 shows, that it also impossible to distinguish between the different complexes (I′, II′, III′) by measuring the FCS signal from the red marker only. In the shown graphs t_{c} means the correlation time and G(t_{c}) the correlation function.

It would be necessary to measure the fluorescence of the green (G) and blue (B) marker additionally, in order to distinguish the beads. This would require complicated optical systems, several lasers for excitation and several detectors. Within the method of the present invention it is possible to use one single excitation laser and one single detector only, to distinguish between the different beads and/or complexes.

1. A method for detecting the binding between binding sites, comprising the following steps:
providing a population of nanoparticles N₁, . . . Nₓ, each nanoparticle having at least one characteristic electrical field property and at least one first binding site, wherein the characteristic electrical field property codes for the corresponding first binding site of each nanoparticle,
adding a sample to be analyzed which possibly comprises at least one analyte with at least one second binding site capable to bind to said first binding site, wherein said analyte and/or said nanoparticles N₁, . . . or Nₓ have at least one detectable property which changes upon binding of said first binding site to said second binding site, the detectable property being different from the electrical field property,
applying an electrical field,
separating said population of nanoparticles with regard to said electrical field property,
measuring the presence or absence of a change of the at least one detectable property.

2. The method according to claim 1, wherein said measurement of the presence or absence of the change of said detectable property is performed simultaneously with or after applying the electrical field.

3. The method according to claim 1 and/or 2, wherein said nanoparticles comprising said first binding site and said analyte comprising said second binding site selectively bind to each other to form a complex, wh rein after said complex formation the presence of a chang of said detectable property can be detected.

4. The method according to at least one of claims 1 to 3, wherein said first binding site comprises an assay component, especially an antigen, an antibody, an enzyme, a receptor, a ligand, DNA, RNA, a receptor, a dendrimer and/or a small peptide.

5. The method according to at least one of claims 1 to 4, wherein each of said nanoparticles N₁, . . . or Nₓ comprises at least one detectable label having said detectable property.

6. The method according to claim 5, wherein one single detectable label is used which is identical for all nanoparticles N₁, . . . or Nₓ.

7. The method according to according to any of claims 1 to 4, wherein each of said analytes comprises at least one detectable label having said detectable property.

8. The method according to claim 7, wherein one single detectable label is used which is identical for all analytes.

9. The method according to at least one of claims 5 to 8, wherein said label is a magnetic label and/or a luminescence label, especially a fluorescence label, and/or a radioactive label.

10. The method according to at least one of claims 1 to 9, wherein said presence or absence of change of said detectable property is measured by detecting radioactivity and/or magnetism and/or absorption and/or colorimetry and/or luminescence, especially fluorescence spectroscopy, in particular by measuring fluorescence intensity (FI) and/or fluorescence polarisation (FP) and/or fluorescence lifetime (FLT), and/or (cc)fluorescence correlation spectroscopy and/or fluorescence intensity distribution analysis (FIDA), 20-FIDA, and/or cFLA and/or FILDA and/or GMR (Giant Magneto Resistance) and/or GMI.

11. The method according to at least one of claims 1 to 10, wherein said nanoparticles N₁, . . . or Nₓ comprise at least one charged molecule which determines their electrical field property and which is bound to the surface of said nanoparticles N₁, . . . or Nₓ.

12. The method according to claim 11, wherein said charged molecule is different from said first binding site and/or said second binding site.

13. The method according to claim 12, wherein said charged molecule is selected from the following group of molecules PO₄³⁻, SO₄²⁻, NH₃⁺, NH₂, DNA or oligonucleotides (permanent and induced dipole moment), highly charged proteins, sugars, starch or organic polymers especially polyamines, polycarboxylates, especially “Babyabsorber”, polyacrylate, PEG backbones polyvinylalcohols, branched polymers, glass and/or polystyrene and/or metaloxide and/or organic polymer and/or supramolecular units, such as dendrimers or fullerenes and/or DNA and/or RNA and/or small peptides.

14. The method according to at least one of claims 11 to 13, wherein a plurality of charged molecules form a shell-like film on the surface of said nanoparticles N₁, . . . or Nₓ.

15. The method according to claim 14, wherein said nanoparticles N₁, . . . or Nₓ are coated by at least two shell-like films which might be the same or different.

16. The method according to at least one of the claims 1 to 15, wherein the electric field property is the susceptibility, and wherein the susceptibility of said nanoparticles N₁, . . . or Nₓ is determined by the number and/or kind of charged molecules.

17. The method according to claim 16, wherein the susceptibility is the AC susceptibility and/or DC susceptibility, and/or AC dielectric susceptibility and/or DC dielectric susceptibility.

18. The method according to at least one of the claims 1 to 17, wherein said electrical field properties of said nanoparticles N₁, . . . or Nₓ change upon binding of the first binding site to the second binding site.

19. The method according to at least one of the claims 1 to 17, wherein said electrical field properties of said nanoparticles N₁, . . . or Nₓ are not, or are only slightly changed upon binding of the first binding site to the second binding site.

20. The method according to at least one of claims 1 to 19, wherein the electrical field exercises forces on said nanoparticles N₁, . . . or Nₓ which result in a net motion of the nanoparticles N₁, . . . or Nₓ relatively to each other.

21. The method according to at least one of claims 1 to 20, wherein at least two nanoparticles N₁, N₂ with different characteristic electrical field properties are used.

22. The method according to at least one of claims 1 to 21, wherein said nanoparticles N₁, . . . or Nₓ are moved by the electrical field forces to an area where in addition to the electrical field, an additional field is applied, preferably a field based on hydrostatic pressure and/or a second electrical field.

23. The method according to at least one of the claims 1 to 22, wherein said det ctable properties of said nanoparticles N₁, . . . or Nₓ are measured while passing along a stationary detector and/or by scanning the individual positions of said nanoparticles N₁, . . . or Nₓ sequentially with a detector and/or simultaneously by using a detector array.

24. The method according to at least one of the claims 1 to 23, wherein said nanoparticles N₁, . . . or Nₓ are nanoparticles with a diameter of less than 10 microns, preferably less than 1 microns, more preferably less than 200 nanometers, most preferably less than 100 nanometers.

25. A nanoparticle showing at least one characteristic electrical field property and at least one first binding site, having a diameter of less than 10 microns, preferably less than 1 microns, more preferably less than 200 nanometers most preferably less than 100 nanometers.

26. The nanoparticle according to claim 25, wherein said nanoparticle comprises glass and/or polystyrene and/or polypropylene and/or metaloxide and/or organic polymer and/or supramolecular units, such as dendrimers or fullerenes and/or DNA and/or RNA and/or small peptides and/or PO₄³⁻, SO₄²⁻, NH₃⁺, NH₂, DNA or oligonucleotides (permanent and induced dipole moment), highly charged proteins, sugars, starch, organic polymers especially polyamines, polycarboxylates, especially “Babyabsorber” polyacrylate, PEG backbones, polyvinylalcohols, branched polymers, dendrimers, or combinations thereof.

27. The nanoparticle according to claim 25 and/or 26, wherein said nanoparticle comprises at least one charged molecule which determines its electrical field property, wherein said charged molecule is different from said first binding site.

28. The nanoparticle according to at least one of claims 23 to 25, wherein said first binding site comprises an assay component, especially an antigen, an antibody, an enzyme, a receptor, a ligand, DNA, RNA, a ligand, a receptor, a dendrimer and/or a small peptide.

29. The nanoparticle according to claim 27, wherein said charged molecule is selected from the following group of molecules PO₄³⁻, SO₄²⁻, NH₃⁺, NH₂, DNA or oligonucleotides (permanent and induced dipole moment), highly charged proteins, sugars starch or organic polymers especially polyamines, polycarboxylates, especially “Babyabsorber”, polyacrylate, PEG backbones, polyvinylalcohols, branched polymers, glass and/or polystyrene and/or metaloxide and/or organic polymer and/or supramolecular units, such as dendrimers or fullerenes and/or DNA and/or RNA and/or small peptides.

30. The nanoparticle according to at least one of claims 25 to 29, wherein a plurality of charged molecules form a shell-like film on the surface of said nanoparticle.

31. The nanoparticle according to at least one-of claims 25 to 30, wherein said nanoparticle is coated by at least two shell-like films, which might be the same or different.

32. The method according to at least one of the claims 25 to 31, wherein said electric field property is the susceptibility, and wherein the susceptibility of said nanoparticle is determined by the number and kind of charged molecules on its surface.

33. The method according to claim 32, wherein said susceptibility is the AC susceptibility and/or DC susceptibility, and/or AC dielectric susceptibility and/or DC dielectric susceptibility.
